(19) United States
(12) Patent Application Publication
(10) Pub. No.: US 2024/0016708 A1
(43) Pub. Date: Jan. 18, 2024

(54) FOAMING CLEANSING COMPOSITIONS

(71) Applicant: DEB IP LIMITED, DENBY, DERBYSHIRE (GB)

(72) Inventors: JOHN HINES, DENBY (GB); MATTHEW SLACK, DENBY (GB); TONY RYAN, DENBY (GB); NIALL WARD-O'BRYAN, SHEFFIELD (GB)

(21) Appl. No.: 18/037,000

(22) PCT Filed: Nov. 16, 2021
(86) PCT No.: PCT/GB2021/052956

§ 371 Date: May 15, 2023

(30) Foreign Application Priority Data
Nov. 17, 2020 (GB) 2018055.0

(51) Int. Cl. A61K 8/04; A61K 8/894; A61Q 19/10

(52) U.S. Cl. CPC A61K 8/046; A61K 8/894; A61Q 19/10; A61K 2800/596; A61K 2800/87; A61K 2800/52

(57) ABSTRACT

The invention provides a method of selecting a polyether polysiloxane surfactant to optimise an alcohol and water containing foaming cleansing composition selecting an amount of alcohol to be used in the final composition; and selecting the degree of polymerisation of the backbone of the siloxane (PDMS) of the polyether polysiloxane surfactant and the degree of polymerisation of polyether side groups of the polyether polysiloxane surfactant for that amount of alcohol from a predetermined optimisation calibration, where the alcohol is a C1 to C4 straight or branched chain alcohol, the polysiloxane backbone is polydimethylsiloxane (PDMS) and the polyether is selected from a polyethylene oxide (PEO), polypropylene oxide (PPO) or a mixed PEO/PPO polyether.

The invention relates to a method of selecting a polyether polysiloxane surfactant to optimise an alcohol and water containing foaming cleansing composition, and optimised foaming compositions, where the alcohol is C1-C4 straight or branched chain alcohol and the polyether is selected from a polyethylene oxide (PEO), polypropylene oxide (PPO), or a mixed PEO/PPO polyether.

Alcohol-containing cleansing skin compositions, such as those used as antibacterial and antiviral cleansing compositions are generally known in the art. Such cleansing compositions typically contain at least 50% volume of the alcohol. Those compositions containing 50% volume alcohol have some antibacterial properties, but are typically less effective at disinfecting viral particles. Typically, the minimum concentration used for personal hand cleansing compositions is 60% volume, to provide adequate antiviral activity. Typically, medical practitioners use formulations containing at least 70% volume alcohol.

One disadvantage of alcohol-containing formulations is that the alcohol often evaporates quickly from the skin. To address such a disadvantage, thickeners have previously been added to such disinfectant solutions in order to increase the viscosity of these agents. This produces disadvantages such as difficulty in dosing due to problems in distributing the thickened formulation evenly and uniformly across the skin or hands.

Production of foamable cleansing formulations using, for example, conventional non-alcohol-containing soaps, is generally known. One problem with simply adding a surfactant to an alcohol-containing formulation has been that the alcohol often inhibits the production of the foam. This requires careful selection of the surfactant in order to produce the desired foaming properties.

WO 2006/066888 and WO 2006/094387 describe high alcohol-containing foaming compositions which utilise silicone-based surfactants. Careful selection of the silicone-based surfactant allowed the alcohol-containing compositions to be foamed, for example using non-pressurised hand pumps.

WO 2006/066888 describes using a surfactant in combination with at least one polyalkylene glycol. The surfactant was selected to have a surface tension of +/−15 dyn/cm of the surface tension of the alcohol component of the formulation. A range of silicone compounds are described in the application, including linear and cyclic silicones and polysiloxane-polyether copolymers. The specially preferred polyether-polysiloxane surfactant is indicated as being a bis-PEG/PPG-20/20 dimethicone, that is a PDMS (polydimethylsiloxane) backbone of indeterminate length comprising a terminal PEO polyether with a number average of 20 residues and a terminal PPO polyether with a number average of 20 residues. This is stated to be used at concentrations between 60 wt % ethanol and 90 wt % ethanol.

WO 2006/09487 again describes the use of a C1-C4 alcohol in concentrations of at least 40% vol/vol of the total composition, in combination with a silicon-based surface active agent in an amount of at least 0.01% of the total composition and water in an amount to balance the total composition to 100%. A variety of silicon-based surfactants are suggested, including bis-PEG-[10-20] dimethicone, 3-(3-hydroxypropyl)-heptamethyltrisilioxane, ethoxylated silicon-based surfactants, bis-PEG/PPG 18/6 dimethicone, a polyether-modified polysiloxane or a polysiloxane betaine. These are suggested across the full range of the concentrations of alcohol described in that published application.

The interaction between siloxane surfactants and their foaming properties in ethanol-water solutions has proved to be complex. The Applicant sought to optimise the siloxane surfactants used within alcohol-containing cleansing formulations. They looked at a number of backbone lengths and polyether side chain lengths and compositions across a range of different alcohol concentrations. The reason for doing this was they found that different siloxanes have different properties, each of which affect whether, for example, the polysiloxane is insoluble in the ethanol-water formulation, whether the siloxane surfactant is able to foam, and whether the siloxane surfactant affects the push speed (speed at which air and the composition may be mixed in a pump-foamer dispenser) to produce the foam. This allows for example the user to develop a foaming formulation that would be robust to changes in the push speed in hand pump dispensers, so that it would not unduly suffer in performance if the pump speed were increased. This therefore allows the formulations to be used in different pumps from different manufacturers or allows for different pump speeds to be used by different end users.

The composition of the polyether chains was also found to affect the properties, depending on the concentration of the alcohol.

The Applicant has now been able to identify those polyether polysiloxanes to select to optimise the foaming of alcohol and water containing foam cleansing compositions for different alcohol formulations.

Accordingly, the invention provides a method of selecting a polyether polysiloxane surfactant to optimise an alcohol and water containing foaming cleansing composition comprising:
(i) selecting an amount of alcohol to be used in the final composition; and
(ii) selecting the degree of polymerisation of the backbone of the siloxane (PDMS) of the polyether polysiloxane surfactant and the degree of polymerisation of polyether side groups of the polyether polysiloxane surfactant for that amount of alcohol from a predetermined optimisation calibration, wherein:
the alcohol is a C1 to C4 straight or branched chain alcohol, the polysiloxane backbone is a polydimethylsiloxane (PDMS) and the polyether is selected from a polyethylene oxide (PEO), polypropylene oxide (PPO) or a mixed PEO/PPO polyether.

The degree of polymer polymerisation of the siloxane backbone means the number average of dimethyl siloxane residues within the backbone.

The degree of polymerisation of the polyether side groups is the number average of PEO and/or PPO residues in each side group attached to the polysiloxane backbone. PEO is also known as polyethylene glycol (PEG). PPO is also known as polypropylene glycol (PPG).

The predetermined calibration allows the polyether polysiloxane(s) to be optimised for a given concentration of the alcohol.

The alcohol may be, for example, methanol, ethanol, propanol, isopropanol or butanol, or mixtures thereof. Most typically the alcohol is ethanol, which may be provided as denatured alcohol. The formulation may comprise a mixture of the alcohols such as a mixture of ethanol and isopropanol. Typically, the formulation comprises at least 50% volume alcohol, 80% or 85% by volume alcohol and may comprise less than 90% volume alcohol. The formulation may comprise a number of additional additives, with water being used to 100% weight of the composition.

The polysiloxane backbone may comprise one or more polyether side groups, for example, 1-45 polyether side groups. Hence, for example, substantially all of the dimethyl siloxane monomers may comprise a polyether backbone. Alternatively, fewer dimethyl siloxane monomers may comprise a polyether backbone, or substantially more dimethyl siloxane monomers may not comprise a polyether backbone.

Typically, 1-20, 1-15, 1-10, 1-5, 1-4, 1-3 or 1 or 2 polyether side groups may be provided for each siloxane backbone.

Typically, the siloxane backbone has a degree of polymerisation (PDMS_{DP}), or comprises 1-50, 2-45, 5-40, 10-30, 15-25 siloxane residues.

Most typically, each siloxane backbone comprises 1 or 2 polyether side groups.

More typically a linear block copolymer is used with one or more typically both ends of the PDMS backbone blocked with PEO and/or PPO, for example as a bis-dimethicone.

Most typically, the number of monomers in the siloxane backbone is between 8 and 45 residues.

The Applicant has unexpectedly identified that mixed PEO/PPO polyethers are preferential in those compositions containing lower amounts of alcohol. Accordingly, when the amount of alcohol is below 70% volume, mixed PEO/PPO polyethers are specially selected. These may be, for example, separate blocks of PEO and PPO as separate polyether side chains. Alternatively, the side chain may each comprise a mixture of PPO and PEO.

Typically, when the amount of alcohol is about 50-less than 70% volume:
(i) the polyether comprises 6-10 monomers, preferably 6-8, per polyether arm; and
(ii) the PDMS_{DP} of the polysiloxane chain is 8-16, preferably 10-12;
or typically the polyether contains 8 monomers and the degree of polymerisation of the PDMS is 10.

Typically, when the amount of alcohol is 70-90% volume:
(I) the polyether comprises at least 10 monomers, preferably 10-18 or 12-16, per polyether arm; and
(ii) the PDMS_{DP} of the polysiloxane chain is greater 16, more typically more than 25, preferably 26-45.

Polyether typically contains 12 monomers and the degree of polymerisation of the PDMS is 30-40.

A further aspect of the invention, when the degree of polymerisation of the polysiloxane backbone is at least 10, or more typically at least 20:
n(TOTALpolyether)≥n(PDMS)^{2/3}×π^{1/3}×36^{1/3}×r
where:
n(TOTALpolyether) is the minimum amount of total polyether side groups. For example where n=16, the PEO groups may be 2 blocks of 8 PEO monomers attached to the PDMS.
n(PDMS) is the degree of polymerisation of the polysiloxane backbone.
r is a constant=0.5

The minimum amount and degree of polymerisation are the number average for the polyether and polysiloxane

Typically, the final concentration comprises the alcohol, at least 0.01 wt % of the final composition of the polyether polysiloxane and water to balance the final composition to 100 wt %. Typically the total amount of the polyether polysiloxane is 1-10 wt %, more typically 5 wt %.

Typically, the composition is foamable when mixed with air at low pressure, for example, at atmospheric pressure or below, wherein when the composition is mixed with air, the mixture of the composition and air forms the foam. Foam dispensing pumps for producing foamed alcohol-water containing formulations are generally known in the art, and are obtainable from, for example, SC Johnson Professional Products Limited, Belper, Derbyshire, United Kingdom. Hand operated pumps, may be used or alternatively automated pumps for foaming the product at low pressure may also be used. The composition may comprise one or more additional surfactants selected from the group consisting of additional silicone-based surface active agents, fluorinated surfactants, alkylglucosides, poly(ethoxylated and/or propoxylated)alcohol, a poly(ethoxylated and/or propoxylated)ester, a derivative of a poly(ethoxylated and/or propoxylated)alcohol, a derivative of a poly(ethoxylated and/or propoxylated)ester, an alkyl alcohol, an alkenyl alcohol, an ester of a polyhydric alcohol, an ether of a polyhydric alcohol, an ester of a polyalkoxylated derivative of a polyhydric alcohol, an ether of a polyalkoxylated derivative of a polyhydric alcohol, a sorbitan fatty add ester, a polyalkoxylated derivative of a sorbitan fatty add ester, a betaine, a sulfobetaine, an imidazoline derivative, an aminoacid derivative, a lecithin, a phosphatide, an amine oxide, a sulfoxide and mixtures thereof, present in an amount between about 0.10% to about 5% weight percent.

The composition may comprise a foam stabiliser at typically between 0 and 5%, more typically 0.01 and 4 wt % of the final composition. The foamed stabilising agent may be selected from the group consisting of lactic acid esters of monoglycerides, cationic emulsifiers, anionic surfactants such as (sodium dodecyl sulphate), quaternary ammonium compounds, triquatemized stearic phospholipid complex, hydroxystearamide propyltriamine salts, lactic acid monoglycerides, food emulsifiers such as glyceryl monostearate, propylene glycol mon[omicron]stearate, sodium stearoyl lactylate, cetyl betaine, glycolether, n-propanol, butyleneglycol, siliconee wax, an encapsulated oil, Microcapsule Mineral Oil, and combinations thereof. The composition may further comprise one or more of a moisturizer, emollient, lipid layer enhancers and combinations thereof selected from the group consisting of lanolin, vinyl alcohol, polyvinyl pyrrolidone and polyols selected from the group consisting of glycerol, propylene glycol, butyleneglycol, glyceryl oleate and sorbitol, cocoglucoside, a fatty alcohol selected from the group consisting of cetyl alcohol, stearyl alcohol, lauryl alcohol, myristyl alcohol and palmityl alcohol, cetyl alcohol, ceteareth 20, an alkylglucoside, mixtures of alkylglucoside and glyceryl Linoleammonium Chloride or PEG-7 Glyceryl Cocoate, and combinations thereof, present in an amount up to about 5%. The pH of the formulation may be adjusted by using an acid or base to adjust the pH of the composition to a pre-selected pH utilising an acid or a base in an amount of to 5% by weight, more typically 0.05-0.5% weight of the total composition and may be citric acid.

The moisturiser may be provided at 0.1-5 wt %.

0.01-5 by wt % of the total composition may be preservative.

The composition may comprise further constituents selected from the group consisting of organic gums and colloids, lower alkanolamides of higher fatty acids, short chain dials and/or triols, fragrance, colouring matter, ultraviolet absorbers, solvents, suspending agents, buffers, conditioning agents, antioxidants, bactericides and medicinally active ingredients, and combinations thereof. The invention further provides compositions obtainable by a method according to the invention.

A still further aspect of the invention provides a foam cleansing composition comprising a polyether polysiloxane surfactant an alcohol and water, wherein the amount of alcohol is about 50-less than 70%% volume, the alcohol is a C1 to C4 straight or branched chain alcohol, mots typically ethanol, and the polyether is selected from a polyethylene oxide (PEO), polypropylene oxide (PPO) or a mixed PEO/PPO polyether; and
(i) the polyether comprises 6-10 monomers, preferably 6-8, per polyether arm; and
(ii) the PDMS_{DP} of the polysiloxane chain is 8-16, preferably 10-12.

The composition typically contains 8 monomers per polyether chain and the PDMS is 10.

A still further aspect of the invention provides a foam cleansing composition comprising a polyether polysiloxane surfactant an alcohol and water, wherein the amount of alcohol is about 70-90% % volume, the alcohol is a C1 to C4 straight or branched chain alcohol, mots typically ethanol, and the polyether is selected from a polyethylene oxide (PEO), polypropylene oxide (PPO) or a mixed PEO/PPO polyether; and
(i) the polyether comprises at least 10 monomers, preferably 10-18 or 12-16, per polyether arm; and
(ii) the PDMS of the polysiloxane chain is greater than 16, typically greater than 25, preferably 26-45.

Typically, the polyether contains 12 monomers and the PDMS is 30-40.
n(TOTALpolyether)≥n(PDMS)^{2/3}×π^{1/3}×36^{1/3}×r
where:
n(TOTALpolyether) is the minimum amount of total polyether side groups n(PDMS) is the degree of polymerisation of the polysiloxane backbone.
r is a constant=0.5

The compositions of the invention may comprise at least 0.01% of the final composition of the polyether polysiloxane and water to balance the composition to 100 wt % (including with the optional components as defined above for the methods of the invention).

Additional components include additional surfactants, foam stabilisers, moisturisers, emollients, lipid layer enhancers and combinations of, acid or bases, preservatives, and further constituents as discussed above for the methods of the invention. Compositions of the invention may be stored in an unpressurised dispenser having a dispenser pump for mixing the air composition with air and dispensing foam therefrom. Typically, the dispenser pump dispenses foam at atmospheric pressure or below.

Computer-implemented methods comprising inputting an amount of alcohol to be used in a composition and selecting a polyether polysiloxane surfactant to optimise an alcohol and water-containing foam cleansing composition by a method of the invention are also provided. The computer may comprise a computer processor and a computer memory.

The components of the compositions are all commercially available or are able to be manufactured by one skilled in the art without undue experimentation.

The invention will now be described by way of example only with reference to the following Figures:

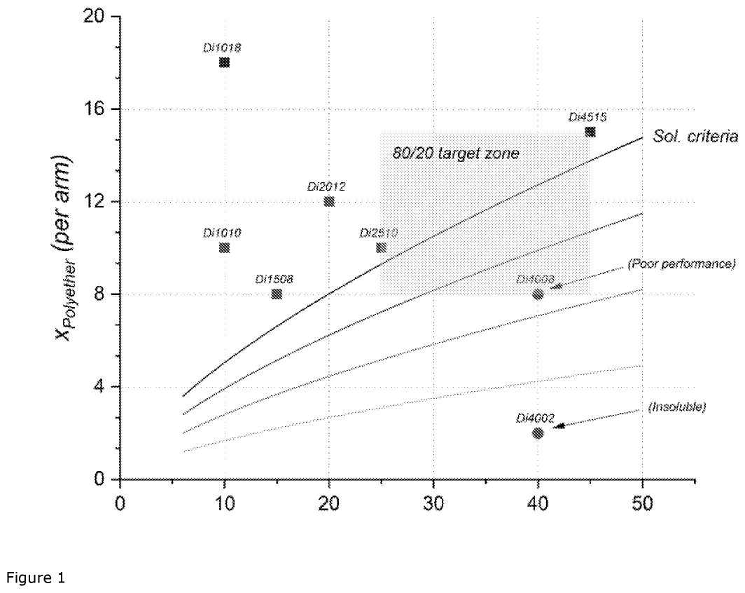

FIG. 1 shows a schematic which describes the range of surfactants tested in this study. Hence for example Di2510 contains two PEO arms (A) of 10 polyether residues per arm and a backbone (B) of 25 PDMS residues. The solubility criteria show predictions for different values of r, as described in the equation above, giving a predicted region of solubility for the range of alcohol concentrations considered.

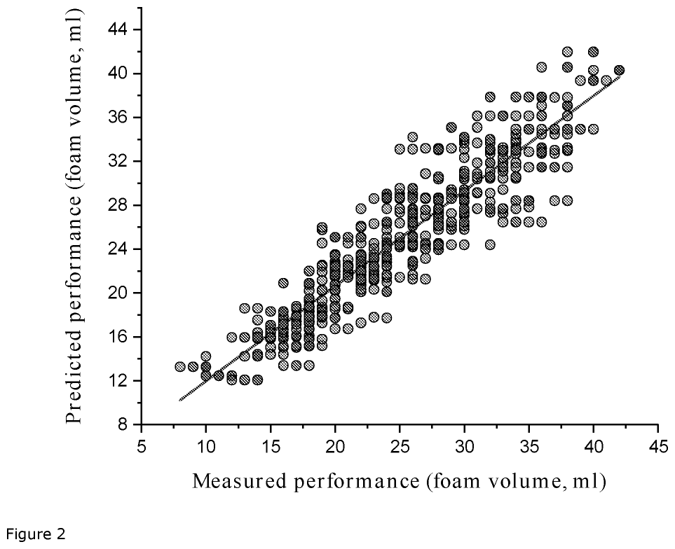

FIG. 2 shows the fit of a refined model compared to experimental data for predicted and actual foam behaviour.

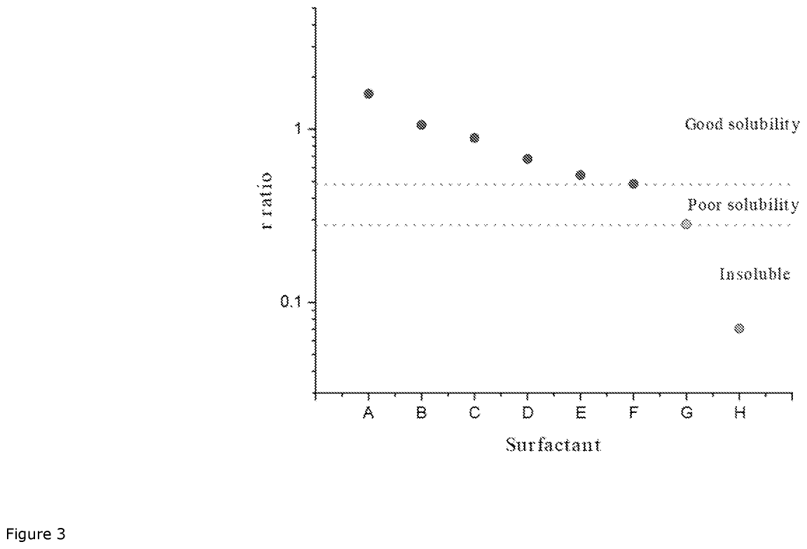

FIG. 3 shows all surfactants with an r value greater than 0.5 have good solubility, whereas those below this limit, particularly with values less than 0.3, have poor solubility or are completely insoluble, leading to poor performance as a surfactant. The compounds A-H are described below.

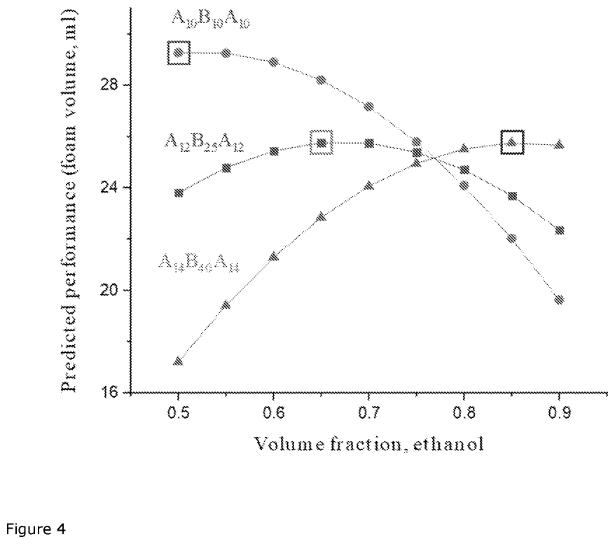

FIG. 4 shows predicted foamabilities for three novel surfactants at fixed weight percentage, across a range of water-alcohol mixtures. Here ‘A’ designates a poly (ethylene oxide) block and B a siloxane block. The formulation within the square represents the optimal water/ethanol mixture for each surfactant.

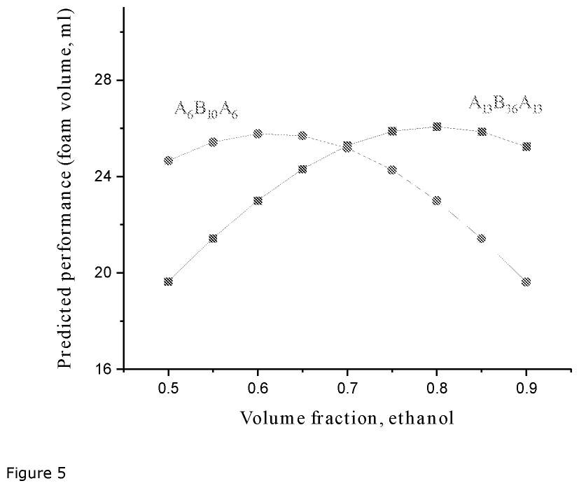

FIG. 5 displays foamability for two sets of surfactant solutions—one optimized for alcohol-poor formulations, and the other for alcohol-rich formulations—at fixed weight percentage

Methods & Experimental

Six triblock siloxane polyethers were investigated, featuring a range of DP_{PDMS} (=10-45) and DP_{Polyether} (=8 to 18 per arm). Two contained polypropylene oxide, or a mixture of polyethylene oxide and polypropylene oxide (A and B)—four had only polyethylene oxide (C-F). A further two siloxane-polyethylene oxide compounds were synthesised by the experimenters (G-H).

These surfactants were foamed by the automated depression of a lever on a commercial hand sanitizer dispenser, which mixed air (˜25 ml) and surfactant solution (1.5 ml) before ejecting them through a mesh. In each measure, the volume of four lever depressions was measured using a conical measuring cylinder and recorded.

Four variables were adjusted—the nature of the surfactant, the surfactant loading, the water-ethanol ratio in the solvent and the time over which the lever was depressed.
A—A₁₈B₁₀A₁₈, where A=PPO
B—A₃₀B₄₅A₃₀, where A=PPO/PEO blend
C—A₁₀B₂₀A₁₀, where A=PEOD—A₁₂B₂₀A₁₂, where A=PEO
E—A₈B₁₅A₈, where A=PEO
F—A₁₀B₂₅A₁₀, where A=PEO
G—A₈B₄₀A₈, where A=PEO
H—A₂B₄₀A₂, where A=PEO

Where Aₓ represents a block of x repeat units of polyether whereas Bₓ represents a block of x repeat units of siloxane. Hence A₁₀B₁₀A₁₀ describes a triblock siloxane polyether, containing one 10-unit block of siloxane, capped with two 10-unit blocks of poly(ethylene) oxide

The surfactants were foamed by the automated depression of a lever on a commercial hand sanitizer dispenser provided by SC Johnson Professional Ltd Denby, Derbyshire UK, which mixed air (˜25 ml) and surfactant solution (1.5 ml) before ejecting them through a mesh. In each measure, the volume of four lever depressions was measured using a conical measuring cylinder and recorded.

The resulting data was analysed by elastic net regression techniques, building a robust model which could be used to predict the foamability of novel siloxane polyether surfactants and determine the ideal surfactant for a given water-alcohol solution. AIC_{C}-based validation to reduce overfitting, and the data was assumed to follow a gamma distribution. All quadratic terms and second order interaction terms were included, except those that suffered from strong multicollinearity with other terms.

FIG. 2 shows that our model achieves good fit to the data, with R²=0.86.

Results

Interactions between solvent quality and surfactant structure

A key feature of the model was the strength of interaction terms linking xₑₜₕ, n_{PDMS} and n_{PEther} The model predicted that a narrow range of n_{PEther} was required for good foamability. The optimal value was strongly dependent on n_{PDMS}—with higher values of n_{PDMS} requiring higher values of n_{pEther} The effect of n_{PDMS}, on the other hand, was strongly dependent on xₑₜₕ—when xₑₜₕ was low, a low value of n_{PDMS} was preferable, and the opposite when xₑₜₕ was high (see FIG. 1).

Influence of Surfactant Concentration

We found that concentration has an important (though marginally diminishing) positive effect on foamability. At the steepest point of the curve, an increase in surfactant concentration by 5 mM was expected to increase foam volume by ˜5 ml. The effect of concentration was moderated by both ethanol fraction and push speed—with higher ethanol content and longer foam generation times making the system more sensitive to concentration.

Influence of Foam Generation Time

The direct effect of push speed was small, accounting for a maximum volume difference of ˜2 ml between the best and worst conditions. We found that the best foamability was typically achieved with moderate values of push speed.

We find that surfactants which are relatively siloxane-rich perform better at high alcohol volume fractions, whereas those which are relatively siloxane-poor perform better at lower alcohol fractions (see figure below).

Here and following on in this document, Aₓ represents a block of x repeat units of poly(ethylene oxide), whereas Bₓ represents a block of x repeat units of siloxane. Hence A₁₀B₁₀A₁₀ describes a triblock siloxane polyether, containing one 10-unit block of siloxane, capped with two 10-unit blocks of poly(ethylene) oxide.

FIG. 4 shows predicted foamabilities for three novel surfactants at fixed weight percentage, across a range of water-alcohol mixtures. Here ‘A’ designates a poly(ethylene oxide) block and B a siloxane block. The formulation within the square represents the optimal water/ethanol mixture for each surfactant.

This data clearly demonstrates the unexpected interaction between surfactant character and solvent characteristics. While A₁₀B₁₀A₁₀ is superior to the displayed alternatives at 50%-70% ethanol, A₁₄B₄₀A₁₄ is clearly superior above 80%. A₁₂B₂₅A₁₂ has relatively robust performance across the range.

Optimization

We combined this model with several optimization procedures, targeting (for instance) the surfactant which, for a given water/alcohol mixture, could produce the maximum volume of foam with a fixed weight inclusion of surfactant. We could also predict which surfactants would produce an adequate amount of foam at the lowest possible loading.

FIG. 5 displays foamability for two sets of surfactant solutions—one optimized for alcohol-poor formulations, and the other for alcohol-rich formulations—at fixed weight percentage. At 70% ethanol by volume, the two surfactants give near-identical performance.

Solubility

Two surfactants, synthesised for use in the study, were found to be partially or completely insoluble in water-ethanol mixtures. This led to the design of a solubility criterion, arising from simple geometric considerations of the surfactant's conformation in solution. The siloxane block is solvophobic and must be screened from solvent interaction by its polyether moieties to remain soluble—it likely forms a spherical globule in solution to minimise surface area. The volume of this globule, V_{PDMS}, scales approximately linearly with the number of siloxane units in the surfactant. If we define V_{PDMS} in polymer-equivalent units—the volume taken up by a siloxane unit—we can define:

$${V}_{SIL} = {n}_{PDMS} = \frac{4}{3} π {r}^{3}$$

The surface area of this globule, S_{PDMS}, can be calculated by rearrangement of the above:

S_{PDMS}=36^{1/3}π^{1/3}n_{PDMS}^{2/3}

By comparing this surface area to the number of polyether units, n_{PEther}, we can calculate a ratio of polyether units per exposed PDMS unit area.

$$r = \frac{{n}_{PEther}}{{S}_{PDMS}}$$

FIG. 3 shows that all surfactants with an r value above 0.5 have good solubility, whereas those below this limit, particularly with values below 0.3, have poor solubility or are completely insoluble, leading to poor performance as a surfactant. This leads to the general formulation:

$${n}_{PEther} \geq ( {36}^{\frac{1}{3}} {π}^{\frac{1}{3}} {n}_{SIL}^{\frac{2}{3}} ) r$$

Where r=0.5 should be used for good solubility and r=0.3 should be used for moderate-low solubility.

1. A method of selecting a polyether polysiloxane surfactant to optimise an alcohol and water containing foaming cleansing composition comprising:
(i) selecting an amount of alcohol to be used in the final composition; and
(ii) selecting the degree of polymerisation of the backbone of the siloxane (PDMS) of the polyether polysiloxane surfactant and the degree of polymerisation of polyether side groups of the polyether polysiloxane surfactant for that amount of alcohol from a predetermined optimisation calibration, wherein:
the alcohol is a C1 to C4 straight or branched chain alcohol, the polysiloxane backbone is polydimethylsiloxane (PDMS) and the polyether is selected from a polyethylene oxide (PEO), polypropylene oxide (PPO) or a mixed PEO/PPO polyether.

2. A method according to claim 1, wherein the formulation comprises at least 50% vol alcohol.

3. A method according to claim 1 wherein the amount of alcohol is less than 90% vol.

4. A method according to claim 1, wherein the alcohol is ethanol.

5. A method according to claim 1, wherein each siloxane backbone comprises 1 to 45 polyether side groups.

6. A method according to claim 5, wherein each siloxane backbone comprises 1 or 2 polyether side groups.

7. A method according to claim 1, wherein, when the amount of alcohol is below 70% vol, mixed PEO/PPO polyethers are preferentially selected.

8. A method according to claim 1, wherein the amount of alcohol is about 50-less than 70%% volume:
(i) the polyether comprises 6-10 monomers, per polyether arm; and
(ii) the PDMS_{DP} of the polysiloxane chain is 8-16.

9. A method according to claim 8 wherein the polyether contains 6-8 monomers and the PDMS_{DP} is 10-12

10. A method according to claim 9, wherein the polyether contains 8 monomers and the PDMS_{DP} is 10.

11. A method according to claim 1, wherein the amount of alcohol is 70-90% volume:
(i) the polyether comprises at least 10 monomers, preferably 10-18 or 12-16, per polyether arm; and
(ii) the PDMS_{DP} of the polysiloxane chain is greater than 25, preferably 26-45.

12. A method according to claim 11, wherein the polyether contains 12 monomers and the PDMS_{DP} is 30-40.

13. A method according to claim 1 wherein, when the degree of polymerisation of the polysiloxane back bone is at least 100:
n(TOTALpolyether)≥n(PDMS)^{2/3}×π^{1/3}×36_{1/3}×r
where:
n(TOTALpolyether) is the minimum amount of total polyether side groups
n(PDMS) is the degree of polymerisation of the polysiloxane backbone.
r is a constant=0.5

14. A method according to claim 1, wherein the final composition comprises the alcohol, at least 0.01 weight percent of the final composition of the polyether polysiloxane and water to balance the final composition to 100 weight percent.

15. The method according to claim 1 wherein the composition is foamable when mixed with air at low pressure, wherein when the composition is mixed with air, the mixture of the composition and air forms a foam.

16. The method according to claim 1, wherein
the composition comprises an additional surfactant is selected from the group consisting of additional silicone-based surface active agents, fluorinated surfactants, alkylglucosides, poly(ethoxylated and/or propoxylated)alcohol, a poly(ethoxylated and/or propoxylated)ester, a derivative of a poly(ethoxylated and/or propoxylated)alcohol, a derivative of a poly(ethoxylated and/or propoxylated)ester, an alkyl alcohol, an alkenyl alcohol, an ester of a polyhydric alcohol, an ether of a polyhydric alcohol, an ester of a polyalkoxylated derivative of a polyhydric alcohol, an ether of a polyalkoxylated derivative of a polyhydric alcohol, a sorbitan fatty acid ester, a polyalkoxylated derivative of a sorbitan fatty acid ester, a betaine, a sulfobetaine, an imidazoline derivative, an aminoacid derivative, a lecithin, a phosphatide, an amine oxide, a sulfoxide and mixtures thereof, present in an amount between about 0.10% to about 5% weight percent.

17. A method according to claim 1 the composition additionally comprising a foam stabiliser.

18. A method according to claim 17, wherein the foam stabilizing agent is selected from the group consisting of lactic acid esters of monoglycerides, cationic emulsifiers, quaternary ammonium compounds, triquatemized stearic phospholipid complex, hydroxystearamide propyltriamine salts, lactic acid monoglycerides, food emulsifiers such as glyceryl monostearate, propylene glycol mon[omicron]stearate, sodium stearoyl lactylate, cetyl betaine, glycolether, n-propanol, butyleneglycol, siliconee wax, an encapsulated oil, Microcapsule Mineral Oil, and combinations thereof.

19. A method according to claim 1 the composition including any one of a moisturizer, emollient, lipid layer enhancers and combinations thereof selected from the group consisting of lanolin, vinyl alcohol, polyvinyl pyrrolidone and polyols selected from the group consisting of glycerol, propylene glycol, butyleneglycol, glyceryl oleate and sorbitol, cocoglucoside, a fatty alcohol selected from the group consisting of cetyl alcohol, stearyl alcohol, lauryl alcohol, myristyl alcohol and palmityl alcohol, cetyl alcohol, ceteareth 20, an alkylglucoside, mixtures of alkylglucoside and glyceryl Linoleammonium Chloride or PEG-7 Glyceryl Cocoate, and combinations thereof, present in an amount up to about 5%.

20. The method according to claim 1, the composition further comprising an acid or a base to adjust a pH of the composition to a preselected pH present in an amount from about 0.05 to about 0.5% weight percent of the total composition.

21. A method according to claim 1, including a preservative in an amount from about 0.01 to about 5% weight percent of the total composition.

22. A method according to claim 1 the composition further comprising constituents selected from the group consisting of organic gums and colloids, lower alkanolamides of higher fatty acids, short chain diols and/or triols, fragrance, colouring matter, ultraviolet absorbers, solvents, suspending agents, buffers, conditioning agents, antioxidants, bactericides and medicinally active ingredients, and combinations thereof.

23. A composition obtainable by a method according to claim 1 stored in an unpressurized dispenser having a dispenser pump for mixing the composition with air and dispensing foam therefrom.

24. A foaming cleansing composition comprising a polyether polysiloxane surfactant an alcohol and water, wherein the amount of alcohol is about 50-less than 70% volume, the alcohol is a C1 to C4 straight or branched chain alcohol, most typically ethanol, and the polyether is selected from a polyethylene oxide (PEO), polypropylene oxide (PPO) or a mixed PEO/PPO polyether; and
the polyether comprises 6-10 monomers, preferably 6-8, per polyether arm; and
(ii) the PDMS_{DP} of the polysiloxane chain is 8-16, preferably 10-12.

25. A composition according to claim 24 wherein the polyether contains 8 monomers and the PDMS_{DP} is 10.

26. A foaming cleansing composition comprising a polyether polysiloxane surfactant an alcohol and water, wherein the amount of alcohol is about 70% to 90% volume, the alcohol is a C1 to C4 straight or branched chain alcohol, mots typically ethanol, and the polyether is selected from a polyethylene oxide (PEO), polypropylene oxide (PPO) or a mixed PEO/PPO polyether; and
the polyether comprises at least 10 monomers, preferably 10-18 or 12-16, per polyether arm; and
(ii) the PDMS_{DP} of the polysiloxane chain is greater than 25, preferably 26-45.

27. A composition according to claim 26, wherein the polyether contains 12 monomers and the PDMS_{DP} is 30-40.

28. A composition according to claim 24 wherein, when the degree of polymerisation of the polysiloxane back bone is at least 10:
n(TOTALpolyether)≥n(PDMS)^{2/3}×π^{1/3}×36^{1/3}×r
where:
n(TOTALpolyether) is the minimum amount of total polyether side groups
n(PDMS) is the degree of polymerisation of the polysiloxane backbone.
r is a constant=0.5

29. A composition according to claim 24, wherein the final composition comprises the alcohol, at least 0.01 weight percent of the final composition of the polyether polysiloxane and water to balance the composition to 100 weight percent.

30. A composition according to claim 24 wherein the composition is foamable when mixed with air at low pressure, wherein when the composition is mixed with air, the mixture of the composition and air forms a foam.

31. A composition according to claim 24, wherein the composition comprises an additional surfactant is selected from the group consisting of additional silicone-based surface active agents, fluorinated surfactants, alkylglucosides, poly(ethoxylated and/or propoxylated)alcohol, a poly(ethoxylated and/or propoxylated)ester, a derivative of a poly(ethoxylated and/or propoxylated)alcohol, a derivative of a poly(ethoxylated and/or propoxylated)ester, an alkyl alcohol, an alkenyl alcohol, an ester of a polyhydric alcohol, an ether of a polyhydric alcohol, an ester of a polyalkoxylated derivative of a polyhydric alcohol, an ether of a polyalkoxylated derivative of a polyhydric alcohol, a sorbitan fatty acid ester, a polyalkoxylated derivative of a sorbitan fatty acid ester, a betaine, a sulfobetaine, an imidazoline derivative, an aminoacid derivative, a lecithin, a phosphatide, an amine oxide, a sulfoxide and mixtures thereof, present in an amount between about 0.10% to about 5% weight percent.

32. A composition according to claim 24 the composition additionally comprising a foam stabiliser.

33. A method according to claim 32, wherein the foam stabilizing agent is selected from the group consisting of lactic acid esters of monoglycerides, cationic emulsifiers, quaternary ammonium compounds, triquatemized stearic phospholipid complex, hydroxystearamide propyltriamine salts, lactic acid monoglycerides, food emulsifiers such as glyceryl monostearate, propylene glycol mon[omicron]stearate, sodium stearoyl lactylate, cetyl betaine, glycolether, n-propanol, butyleneglycol, siliconee wax, an encapsulated oil, Microcapsule Mineral Oil, and combinations thereof.

34. A composition according to claim 24 the composition including any one of a moisturizer, emollient, lipid layer enhancers and combinations thereof selected from the group consisting of lanolin, vinyl alcohol, polyvinyl pyrrolidone and polyols selected from the group consisting of glycerol, propylene glycol, butyleneglycol, glyceryl oleate and sorbitol, cocoglucoside, a fatty alcohol selected from the group consisting of cetyl alcohol, stearyl alcohol, lauryl alcohol, myristyl alcohol and palmityl alcohol, cetyl alcohol, ceteareth 20, an alkylglucoside, mixtures of alkylglucoside and glyceryl Linoleammonium Chloride or PEG-7 Glyceryl Cocoate, and combinations thereof, present in an amount up to about 5%.

35. A composition according to claim 24, the composition further comprising an add or a base to adjust a pH of the composition to a preselected pH present in an amount from about 0.05 to about 0.5% weight percent of the total composition.

36. A composition according to claim 24, the composition according to any one of claims 1 to 20 including a preservative in an amount from about 0.01 to about 5% weight percent of the total composition.

37. A composition according to claim 24, the composition further comprising constituents selected from the group consisting of organic gums and colloids, lower alkanolamides of higher fatty acids, short chain diols and/or triols, fragrance, colouring matter, ultraviolet absorbers, solvents, suspending agents, buffers, conditioning agents, antioxidants, bactericides and medicinally active ingredients, and combinations thereof.

38. A composition according to claim 24 stored in an unpressurized dispenser having a dispenser pump for mixing the composition with air and dispensing foam therefrom.

39. A computer implemented method comprising imputing an amount of alcohol to be used in a composition and selecting a polyether polysiloxane surfactant to optimise an alcohol and water containing foaming cleansing composition by a method according to claim 1.

40. A method according to claim 39, wherein the computer comprises a computer processor and a computer memory.
